(19) United States
(12) Patent Application Publication
(10) Pub. No.: US 2006/0100593 A1
(43) Pub. Date: May 11, 2006

(54) Disposable nonwoven undergarments for administering a medicinal agent

(76) Inventor: Clifford Bridges, Greensboro, NC (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/213,394

(22) Filed: Aug. 26, 2005

Related U.S. Application Data
(60) Provisional application No. 60/604,629, filed on Aug. 26, 2004.

(51) Int. Cl. A61M 35/00

(52) U.S. Cl. 604/289

(57) ABSTRACT

A disposable nonwoven undergarment that includes a medicinal agent for the treatment of various medical conditions that affect the lower torso. In accordance with the present invention, a disposable nonwoven undergarment is imparted with a medicinal agent to conveniently administer a medicinal agent to the skin via normal contact, wearer motion, and/or body heat. Such undergarments are suitable for men, women, and children of all ages and especially suitable for those individuals with busy, active life-styles.

### CROSS-REFERENCE TO RELATED APPLICATION

This application claims the benefit of priority Provisional Application No. 60/604,629, filed Aug. 26, 2004, the disclosures of which is incorporated herein by reference.

### FIELD OF THE INVENTION

The present invention relates to a disposable nonwoven undergarment and, more particularly, to a disposable nonwoven undergarment that includes a medicinal agent for the treatment of various medical conditions.

### BACKGROUND OF THE INVENTION

Common symptoms of skin infections that are often treated by way of a topical ointment, cream, or salve, include dryness, redness, burning, and itching. Gels and powders are used as well to address skin inflammation caused by the combination of heat and moisture breaking down the pH balance of the skin. Further, some hormonal therapy regiments utilize a systemic prescription along with a topical gel for a desired effect.

The aforementioned medicinal agents can be used for prolonged lengths of time and may require repeat applications throughout the day. Handling the ointments, creams, and powders can be messy and unpleasant. Ones privacy can be compromised when utilizing such creams and powders in public facilities, such as a fitness centers, school locker rooms, or restrooms, due a strong odor that may be associated with a cream or mishandling of the container that may result in spilling. In addition, it can become burdensome to remember to repeat applications throughout the day.

It is known in the art to impart skin conditioning agents into absorbent articles, such as baby diapers and adult diapers to reduce the chances of developing a rash induced by urine or feces irritated skin. United States Patent Publication No. 2003/0195486, entitled “Absorbent Article having a Stable Skin Care Composition”, published Oct. 16, 2003, in the name of inventors Gatto et al., is representative of such an absorbent article. However, these articles are multi-layered structures specifically suited to be absorbent and to retain urine and/feces. Absorbent pads have also been utilized to administer a pharmaceutical substance, for example, as disclosed in United States Patent Publication No. 2002/0193758, entitled “Product”, published Dec. 19, 2002, in the name of inventor Sandberg.

Although disposable absorbent “diaper-like” articles and pads are widely accepted due to the convenience of use, such articles are often cumbersome, uncomfortable, noisy, and aesthetically unappealing. A need remains for a disposable undergarment that can perform as a vehicle to administer a medicinal agent to the skin, while at the same time provide a soft, form-fitting feel and is aesthetically pleasing, offering the wearer reassuring comfort.

### SUMMARY OF THE INVENTION

The present invention relates to a soft feeling, aesthetically pleasing, and form-fitting disposable nonwoven undergarment that includes a medicinal agent for the treatment of various medical conditions that affect the lower torso.

In accordance with the present invention, a disposable nonwoven undergarment is imparted with a medicinal agent to conveniently administer a medicinal agent to the skin via normal contact, wearer motion, and/or body heat. Such undergarments are suitable for men, women, and children of all ages and especially suitable for those individuals with busy, active life-styles. The disposable nonwoven undergarments are durable, conform to the body, and allow the individual to comfortably engage in sports and travel. The undergarments further maintain an individual's privacy, as the medicinal purpose of the undergarment isn't readily noticeable, the undergarment is aesthetically appealing, easily removed, and easily disposed of.

The disposable undergarment typically comprises a medicinal agent in the crotch region to come in contact with and treat the inflamed skin area. It has also been contemplated to incorporate a medicinal agent in more than one region of the undergarment. The undergarment of the present invention may be utilized as a post-treatment to laser hair removal surgeries or spa hair removal treatments. In this instance, a soothing and cooling gel may be imparted along the frontal region of the undergarment as well as the crotch region to provide cooling relief to sensitive and irritated skin.

Individuals with a mild incontinence condition, who require a device suitable for limited urinary containment, may also benefit from an undergarment made in accordance with the present invention. The disposable nonwoven undergarment may optionally be equipped with a moisture management panel.

In one embodiment of the invention, a disposable nonwoven fabric undergarment article for administering one or more medicinal agents is defined. The undergarment includes a front panel formed of the nonwoven fabric, a back panel formed of the nonwoven fabric, and a crotch region formed of the nonwoven fabric that interconnects the front panel and the back panel to form a waist opening and two leg openings. In addition, at least a portion of the nonwoven fabric is imparted with at least one medicinal agent.

The nonwoven fabric may additionally include one or more three-dimensional images, apertures and/or patterns. The non woven fabric may include synthetic fibers, natural fibers or a blend of synthetic and natural fibers. The synthetic fibers may include polyacrylates or thermoplastic polymers, such as polyamides, polyesters, polyolefins, their derivatives, combinations thereof and the like. The natural fibers may include cotton, wood pulp, rayon and the like.

The medicinal agent may include, but are not limited to any known, anticipated, or otherwise perceived medicinal agent, such as pharmaceuticals, such as antibiotics and anti-inflammatories, and holistic agents, such as essential oils, teas, and other aromatics. The medicinal agent may be imparted into the fibers or it may be applied topically to the surface of the fabric. Typically, in some embodiments the medicinal agent will be limited to the crotch region of the undergarment although other regions are also contemplated. In those embodiment in which the fabric includes images, the portion of the fabric that includes the medicinal agent may be limited to the imaged portions.

In another embodiment a process is defined for fabricating a disposable nonwoven fabric undergarment for administering a medicinal agent. The process includes the steps of providing a precursor nonwoven web, hydroentangling the precursor nonwoven web on an image transfer device to form a patterned and imaged nonwoven fabric, forming the patterned and imaged nonwoven fabric into a front panel, a back panel, and an interconnecting crotch region and joining the front panel, the back panel, and the interconnecting crotch region to form a disposable undergarment. The process also includes the step of incorporating a medicinal agent into the disposable undergarment.

Other features and advantages of the present invention will become readily apparent from the following detailed description, the accompanying drawings, and the appended claims.

### BRIEF DESCRIPTION OF THE DRAWINGS

Having thus described the invention in general terms, reference will now be made to the accompanying drawings, which are not necessarily drawn to scale, and wherein:

FIG. 1 is a perspective view of a “brief” style disposable undergarment according to the present invention;

FIG. 2 is a perspective view of a male “bikini” style disposable undergarment according to the present invention; and

FIG. 3 is a depiction of the apparatus for the fabrication of the nonwoven fabric, in accordance with an embodiment of the present invention

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout

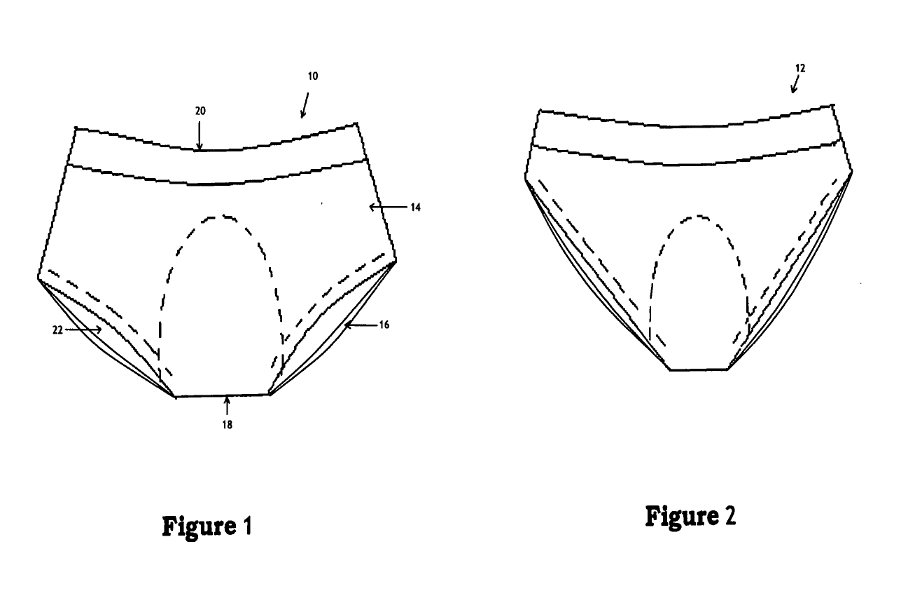

In accordance with an embodiment of the present invention a disposable non-woven fabric undergarment article is defined. FIGS. 1 and 2 illustrate a front-view perspective of a disposable non-woven fabric undergarment, in accordance with the present invention. FIG. 1 illustrates a conventional “brief” type undergarment 10 and FIG. 2 illustrates a conventional “bikini” type undergarment 12. The undergarment includes a front panel 14 formed of the non-woven fabric, a back panel 16 formed of the non-woven fabric, and a crotch region 18 that interconnects the front and back panels to form a waist opening 20 and two leg openings 22. At least a portion of the non-woven fabric is imparted with a medicinal agent (not shown in FIGS. 1 and 2). In the illustrated embodiment the medicinal agent is imparted in an interior portion of the crotch region, although the medical agent can be imparted in any other portion or throughout the entire article.

The one or more medicinal agents in one or more regions of the undergarment will typically provide for the treatment of ailments that affect the lower torso of the body, including the hips, buttock, groin area and the like. The medicinal agent may be imparted to the fibers of the non-woven fabric, applied topically to the surface of the non-woven fabric or imparted in any other suitable manner. The medicinal agents may include, but are not limited to any known, anticipated, or otherwise perceived medicinal agent, such as pharmaceuticals, such as antibiotics and anti-inflammatories, and holistic agents, such as essential oils, teas, and other aromatics. The medicinal agents may be incorporated in any form, such as, but not limited to, aqueous, gel, wax, powder form or the like. Furthermore, the medicinal agents may be imparted in or around fibrous components of the undergarment fabric. When the fabric of the undergarment is a laminate fabric, the medicinal agent may be incorporated in a single layer or all the layers. The treated undergarment regions may be covered with a film for packaging purposes, wherein the film is removed prior to use as to expose the medicinal agent.

The front panel, back panel and crotch region may be interconnected by any suitable connecting means, such as adhesively bonded, mechanically stitched, thermally welded or the like. Typically the panels and crotch region will be secured together at their transverse edges. The nonwoven undergarment may further include two elasticized leg openings and an elasticized waist.

The nonwoven fabric used in accordance with the present invention will typically be durable and able to withstand the stress of continual body movement involved with everyday activities. The fabric will typically also have suitable drape and hand properties so that the fabric will conform to the curves of the body. In addition, the fabric will typically have a hydrophilic side and a hydrophobic side in order absorb and repel moisture where needed.

The nonwoven fabric included in the disposable undergarment may include, but are not limited to, natural fibers, synthetic fibers, or a combination of natural and synthetic fibers. The fibers may be of finite staple length, continuous filaments and the blends thereof. Synthetic fibers may be selected from thermoset polymers such as polyacrylates, or from thermoplastic polymers, including; polyamides, polyesters, or polyolefins, such as polypropylene or polyethylene, their derivatives, and combinations thereof. The synthetic fibers of the present invention may also include any fibers with multi-component configurations, such as side-by-side or sheath-core, as well as geometric variations. The natural fibers of the present invention may be cellulosic in nature, such as cotton, wood pulp, rayon or the like.

The nonwoven fabric of the present invention may be a composite, laminate, single layer or multiple layers in order to incorporate support and/or absorbent mechanisms into the undergarment. Suitable layers that may be utilized include, but are not limited to films, both microporous and monolithic, apertured films, embossed film, continuous filament fabrics, nano-denier fabrics, and additional carded fabrics. The nonwoven may be imaged, such as with ribs or swirls, etc., aperture, such that the apertures extend either partially or fully through the fabric, or modified aesthetically through subsequent dyeing, and printing, or by using colored fibers during the manufacturing step, to achieve the affects of the desired nonwoven disposable undergarment. The nonwoven fabric has a preferred basis weight range of 1.5-5.5 ounces per square yard, with a range of 2.0-3.0 ounces per square yard being most preferred.

The nonwoven fabric may additionally include patterns imparted on a portion or the entirety of the fabric or images, such as three-dimensional images, embossed on a portion or the entirety of the fabric. In such embodiments it may be desirable to incorporate the medicinal agent in the regions that include the pattern or the image.

A disposable nonwoven undergarment has skin health and hygiene benefits. The breathable and moisture absorbing characteristics of the nonwoven fabric utilized in the disposable undergarment provides the skin with a comfortable environment. The nonwoven fabric helps keep the skin oxygenated and dry, which is beneficial for those individuals with an active lifestyle.

In one exemplary embodiment the non-woven fabric may include 50% polyester and 50% rayon arranged in a layered construct such that a soft, hydrophilic rayon fabric layer is formed in the interior of the undergarment, i.e., against the skin, and a hydrophobic, polyester fabric layer forms the exterior of the undergarment. In addition, this exemplary embodiment, as well as other embodiments of the undergarment, may include a discrete cellulosic protective panel for moisture management.

The nonwoven disposable undergarment is typically of the representative “brief” type design shown in FIG. 1 or the “bikini” type design of FIG. 2. Although other types of undergarment designs, such as “athletic brief”, “boxer” or the like are also contemplated and within the scope of the present invention. However, for the sake of insuring that the medicinal agent properly interacts with the skin, the most advantageous embodiments of undergarments will take rest proximate the skin of the wearer. The undergarment of the present invention may be scaled to various sizes, i.e., small, medium, large, etc, in order to accommodate various body types or forms. The undergarment may optionally contain an elastic portion around the waist and leg openings.

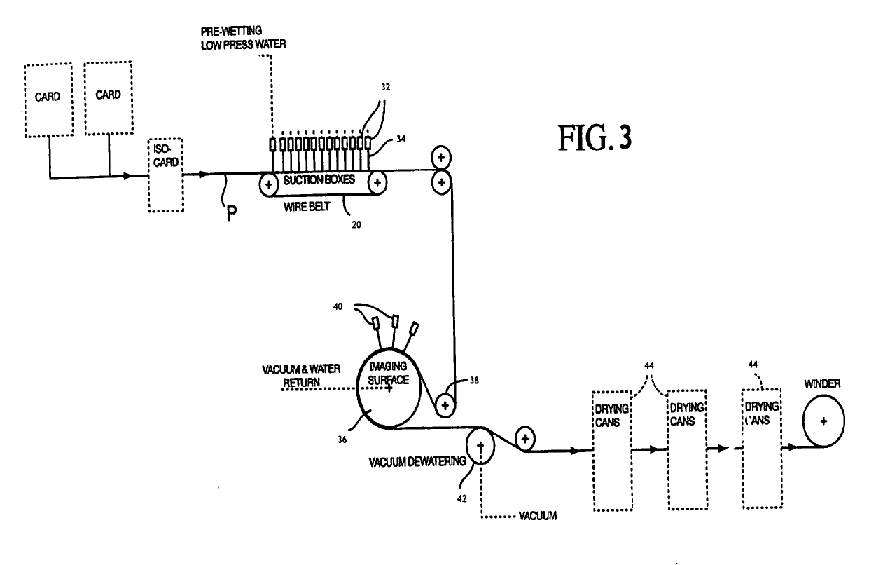

FIG. 3, therein is illustrated an apparatus for practicing the method of the present invention for forming a nonwoven fabric. The fabric is formed from a fibrous matrix, which comprises fibers selected to promote economical manufacture, while achieving the desired resultant nonwoven fabric. The fibrous matrix is preferably carded and subsequently aid-randomized to form a precursor web, designated P.

FIG. 1 illustrates a hydroentangling apparatus for forming nonwoven fabrics in accordance with the present invention. The apparatus includes a foraminous forming surface in the form of a flat bed entangler 30 upon which the precursor web P is positioned for pre-entangling. Precursor web P is then sequentially passed under entangling manifolds 32, whereby the precursor web is subjected to high-pressure water jets 34. This process is well known to those skilled in the art and is generally taught by U.S. Pat. No. 3,485,706, to Evans, hereby herein incorporated by reference as if set forth fully herein.

The entangling apparatus of FIG. 1 further includes an optional imaging and patterning drum 36 comprising a three-dimensional image transfer device for effecting imaging and patterning of the now-entangled precursor web. After pre-entangling, the precursor web is trained over a guide roller 38 and directed to the image transfer device 36, where a three-dimensional image is imparted into the fabric on the foraminous forming surface of the device. The web of fibers is juxtaposed to the image transfer device 36, and high pressure water from manifolds 40 is directed against the outwardly facing surface from jet spaced radially outwardly of the image transfer device 36. The image transfer device 36, and manifolds 40, may be formed and operated in accordance with the teachings of commonly assigned U.S. Pat. No. 4,098,764, No. 5,244,711, No. 5,822,823, and No. 5,827,597, the disclosures of which are hereby incorporated by reference. It is presently preferred that the precursor web P be given a three-dimensional image suitable to provide fluid management, as will be further described, to promote use of the present nonwoven fabric in disposable absorbent articles. The entangled fabric may be, optionally, vacuum dewatered at 42, and dries at an elevated temperature on drying cans 44.

Thus, the present invention provides for a soft feeling, aesthetically pleasing, and form-fitting disposable nonwoven undergarment that includes a medicinal agent for the treatment of various medical conditions that affect the lower torso.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A disposable nonwoven fabric undergarment article for administering one or more medicinal agents:
a front panel formed of the nonwoven fabric;
a back panel formed of the nonwoven fabric, and a crotch region formed of the nonwoven fabric that interconnects the front panel and the back panel to form a waist opening and two leg openings,
wherein at least a portion of the nonwoven fabric is imparted with at least one medicinal agent.

2. The article of claim 1, wherein the nonwoven fabric includes one or more three-dimensional images.

3. The article of claim 1, wherein the nonwoven fabric includes one or more apertures.

4. The article of claim 1, wherein the nonwoven fabric includes one or more patterns.

5. The article of claim 1, wherein the at least one medicinal agent is administered to the skin via normal contact, wearer motion, or body heat.

6. The article of claim 1, wherein the nonwoven fabric is a fibrous blend of synthetic and natural fibers.

7. The article of claim 6, wherein the synthetic fibers are further defined as synthetic fibers chosen from the group consisting of polyacrylates and thermoplastic polymers.

8. The article of claim 7, wherein the thermoplastic polymers are further defined as chosen from the group consisting of polyamides, polyesters, polyolefins, their derivatives, and combinations thereof.

9. The article of claim 6, wherein the synthetic fibers are polyester.

10. The article of claim 6, wherein the natural fibers are selected from the group consisting of cotton, wood pulp and rayon.

11. The article of claim 1, wherein the portion of the nonwoven fabric that is imparted with at least one medicinal agent further defines the portion as the crotch region.

12. The article of claim 1, wherein the front panel further comprises one or more imaged portions.

13. The article of claim 12, wherein the portion of the nonwoven fabric that is imparted with at least one medicinal agent further defines the portion as the one or imaged portions of the front panel.

14. The article of claim 1, wherein the back panel further comprises one or more imaged portions.

15. The article of claim 14, wherein the portion of the nonwoven fabric that is imparted with at least one medicinal agent further defines the portion as the one or imaged portions of the back panel.

16. The article of claim 1, wherein the crotch region further comprises one or more imaged portions.

17. The article of claim 16, wherein the portion of the nonwoven fabric that is imparted with at least one medicinal agent further defines the portion as the one or imaged portions of the crotch region.

18. The article of claim 1, wherein the front panel is further defined as being patterned.

19. The article of claim 1, wherein the back panel is further defined as being patterned.

20. The article of claim 1, wherein the crotch region is further defined as being patterned.

21. The article of claim 1, further comprising a moisture absorbent panel proximate the crotch region.

22. The article of claim 1, further comprising two elastically lined leg openings and an elastically lined waist opening.

23. A process for fabricating a disposable nonwoven fabric undergarment for administering a medicinal agent, the process comprising the steps of:
providing a precursor nonwoven web;
hydroentangling the precursor nonwoven web on an image transfer device to form a patterned and imaged nonwoven fabric;
forming the patterned and imaged nonwoven fabric into a front panel, a back panel, and an interconnecting crotch region;
joining the front panel, the back panel, and the interconnecting crotch region to form a disposable undergarment; and
incorporating a medicinal agent into the disposable undergarment.

24. The process of claim 23, wherein the step of providing a precursor nonwoven web further comprises providing a precursor nonwoven web that is a fibrous blend of synthetic and natural fibers.

25. The process of claim 24, wherein the step of providing a precursor nonwoven web that is a fibrous blend of synthetic and natural fibers further comprises providing a precursor nonwoven web that is a fibrous blend of synthetic and natural fibers, wherein the synthetic fibers are chosen from the group consisting of polyacrylates and thermoplastic polymers.

26. The process of claim 25, wherein step of the providing a precursor nonwoven web that is a fibrous blend of synthetic and natural fibers, wherein the synthetic fibers are chosen from the group consisting of polyacrylates and thermoplastic polymers and the thermoplastic polymers are chosen from the group consisting of polyamides, polyesters, polyolefins, their derivatives, and combinations thereof.

27. The process of claim 24, wherein the step of providing a precursor nonwoven web that is a fibrous blend of synthetic and natural fibers further comprises providing a precursor nonwoven web that is a fibrous blend of synthetic and natural fibers, , wherein the synthetic fibers are polyester.

28. The process of claim 24, wherein the step of providing a precursor nonwoven web that is a fibrous blend of synthetic and natural fibers further comprises providing a precursor nonwoven web that is a fibrous blend of synthetic and natural fibers, wherein the natural fibers are chosen from the group consisting of cotton, wood pulp, and rayon.

29. The process of claim 23, wherein the step of hydroentangling the precursor nonwoven web on an image transfer device to form a patterned and imaged nonwoven fabric further comprises the step of hydroentangling the precursor nonwoven web on an image transfer device to form a patterned and three-dimensional image on the nonwoven fabric.

30. The process of claim 23, wherein the step of joining the front panel, the back panel, and the interconnecting crotch region to form a disposable undergarment further comprises welding thermally the front panel, back panel, and the interconnecting crotch region to form a disposable undergarment.

31. The process of claim 23, wherein the step of joining the front panel, the back panel, and the interconnecting crotch region to form a disposable undergarment further comprises bonding adhesively the front panel, back panel, and the interconnecting crotch region to form a disposable undergarment.

32. The process of claim 23, wherein the step of joining the front panel, the back panel, and the interconnecting crotch region to form a disposable undergarment further comprises stitching mechanically the front panel, back panel, and the interconnecting crotch region to form a disposable undergarment.

33. The process of claim 23, wherein the step of incorporating a medicinal agent into the disposable undergarment further comprises incorporating the medicinal agent into the fibers of the disposable undergarment.

34. The process of claim 23, wherein the step of incorporating a medicinal agent into the disposable undergarment further comprises incorporating a medicinal agent onto the surface of the fibers of the disposable undergarment.

35. The process of claim 23, wherein the step of incorporating a medicinal agent into the disposable undergarment further comprises incorporating a medicinal agent into the crotch region of the disposable undergarment.
